(19) United States
(12) Patent Application Publication
(10) Pub. No.: US 2016/0367461 A1
(43) Pub. Date: Dec. 22, 2016

(54) COSMETIC ANTIDANDRUFF COMPOSITION BASED ON ELLAGIC ACID OR A DERIVATIVE THEREOF AND A SECOND, DIFFERENT ACTIVE COMPOUND IN A SPECIFIC WEIGHT RATIO

(71) Applicant: L'OREAL, Paris (FR)

(72) Inventor: Tiphaine Derkx, Paris (FR)

(21) Appl. No.: 15/253,388

(22) Filed: Aug. 31, 2016

Related U.S. Application Data
(62) Division of application No. 13/582,718, filed on Jan. 17, 2013.
(60) Provisional application No. 61/313,855, filed on Mar. 15, 2010.

(30) Foreign Application Priority Data
Mar. 1, 2010 (FR) 1051446

(51) Int. Cl. A61K 8/49; A61K 8/23; A61K 8/04; A61Q 5/00; A61Q 5/02

(52) U.S. Cl. CPC A61K 8/498; A61Q 5/006; A61Q 5/02; A61K 8/042; A61K 8/4933; A61K 8/4926; A61K 8/23

(57) ABSTRACT

The invention relates to a cosmetic composition comprising, in a cosmetically acceptable medium: (1) one or more compounds selected from ellagic acid, its ethers an the salts of ellagic acid and its ethers, and (ii) one or more compounds selected from pyrithione salts, 1-hydroxy-2-pyridone derivates and selenium (poly)sulphides, in a weight ratio of the amount of active(s) (i) to the amount of antidandruff agent(s) (ii) of greater than or equal to 0.5. The invention also relates to a method of cosmetic treatment of employing such a composition, and to the use of this composition for the treatment of dandruff conditions and for reducing the phenomena of irritation and itching.

### CROSS REFERENCE TO RELATED APPLICATION

This application is a divisional application of U.S. application Ser. No. 13/582,718, filed Jan. 17, 2013, which is a national phase application based on PCT/EP2011/053015 filed Mar. 1, 2011, which claims priority from French Application No. 1051446, filed Mar. 1, 2010, and claims the benefit of U.S. Provisional Application No. 61/313,855, filed on Mar. 15, 2010, the content of all of which is incorporated herein by reference in their entirety.

### TECHNICAL FIELD

The present invention relates to a cosmetic composition comprising at least one first compound selected from ellagic acid, its ethers, the salts of these compounds, and mixtures thereof, and at least one second antidandruff compound, in specific proportions; to a method of antidandruff treatment that employs the said composition; and to the use of the said composition for treating dandruff conditions, by eliminating and/or reducing, in particular, irritations and/or itching on the scalp. The invention relates also, in particular, to the use of the combination of at least one first compound selected from ellagic acid, its ethers and the salts of these compounds and at least one specific second compound, in specific proportions, as an antidandruff agent.

### BACKGROUND

The problems of dandruff affect up to 50% of the world population. They affect both men and women and are perceived as having a very negative psychosocial impact. The appearance of dandruff is disagreeable both aesthetically and because of the inconveniences it causes (itching, redness, etc.), and as such many people confronted with this problem to variable degrees wish to eliminate it efficiently and definitively.

Dandruff corresponds to excessive and visible desquamation of the scalp, resulting from excessively rapid multiplication of the epidermal cells and their abnormal maturation. This phenomenon can be caused in particular by microtraumas of physical or chemical nature, such as hair treatments which are too aggressive, extreme climatic conditions, nervousness, diet, fatigue or pollution, but it has been demonstrated that dandruff conditions most commonly result from a disorder of the microflora of the scalp, and more particularly from excessive colonization by a fungus which belongs to the family of yeasts of the Malassezia genus (formerly known as Pytirosporum) and which is naturally present on the scalp.

To treat dandruff, it is known practice to use antidandruff agents, especially antifungal and/or antibacterial agents in a medium capable of distributing these agents and depositing them on the integuments.

Among antidandruff agents, the use of zinc pyrithione, piroctone olamine or selenium disulphide has been most particularly recommended on account of their powerful cytostatic activity. Although they are highly effective against dandruff, their frequent use has the disadvantage of exacerbating the sensations of discomfort (itching, stinging, hotness, redness or sensations of dryness). Moreover, these antidandruff agents are not entirely satisfactory in terms of their impact on the environment. Fuel supply and delivery systems for vehicles with internal combustion engines are widely known today. Numerous improvements have been developed and put into effect over time to improve the basic systems.

### SUMMARY OF THE INVENTION

Accordingly, there exists a need on the part of users of antidandruff treatments for compositions which exhibit better skin tolerance while retaining high antidandruff efficacy, and which are more environmentally friendly. In addition, antidandruff compositions that are stable and that have good working properties, especially during the application or removal of the compositions, are sought.

The applicant has now found, surprisingly, that the use of at least one first compound selected from ellagic acid, its ethers, salts of ellagic acid, salts of its ethers, and mixtures of these compounds, and of at least one second, different compound selected from specific antidandruff agents, in a specific weight ratio, is able to meet such a need and to overcome the disadvantages of the prior art.

The composition according to the invention allows more effective reduction and/or elimination of dandruff on the hair and scalp, especially that caused by yeasts of the genus Malassezia, whilst substantially reducing the phenomena of irritation and itching of the scalp.

The composition, moreover, does not adversely alter the cosmetic properties of the hair which is treated at the same time, particularly with regard to feel.

It also has a satisfactory stability on storage both at ambient temperature (25° C.) and at elevated temperature (37 or 45° C., for example).

The present invention accordingly provides a cosmetic composition comprising:

(i) one or more compounds selected from ellagic acid, its ethers and the salts of ellagic acid and its ethers, and

(ii) one or more compounds selected from pyrithione salts, 1-hydroxy-2-pyridone derivatives and selenium (poly)sulphides, in a weight ratio of the amount of compound(s) (i) to the amount of compound(s) (ii) of greater than or equal to 0.5.

Further provided by the invention is a cosmetic antidandruff treatment method intended to eliminate and/or reduce dandruff, especially that caused by yeasts of the genus Malassezia, which employs the said composition.

The invention further provides for the use of the said composition for treating dandruff conditions, in other words for eliminating and/or reducing the level of dandruff on the hair and the scalp, and eliminating and/or reducing the phenomena of irritation and/or itching of the scalp.

Other features, aspects, subjects and advantages of the present invention will emerge more clearly from a reading of the description and examples which follow.

### DESCRIPTION OF THE PREFERRED EMBODIMENTS

The composition according to the invention comprises one or more compounds (i) selected from ellagic acid, its ethers, the salts of ellagic acid and the salts of its ethers.

Ellagic acid, also known as 2,3,7,8-tetrahydroxy-1-benzopyrano[5,4,3-cde]-1-benzopyran-5,10-dione, is a well-known molecule which is present in the plant kingdom. Reference may be made to the published Merck Index, 20th edition (1996), No. 3588.

Ellagic acid has the following chemical formula:

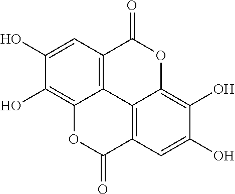 [SMILES: O=c1oc2c(O)c(O)cc3c(=O)oc4c(O)c(O)cc1c4c23]

which comprises four fused rings.

Ellagic acid is commercially available, especially from the company Sigma, France.

Document FR-A-1 478 523 discloses a process for purifying ellagic acid and also the purified ellagic acids obtained via such a process.

The ether or ethers of ellagic acid which can be used according to the invention are preferably selected from the mono-, di-, tri- or polyethers obtained by etherification of one or more hydroxyl groups (one of the four OH groups of ellagic acid) of ellagic acid to one or more groups OR, R being selected from C₂-C₂₀ alkyl groups, polyoxyalkylene groups, and more particularly polyoxyethylene and/or polyoxypropylene groups, and groups derived from one or more mono- or polysaccharides, such as, for example, the group of the formula below:

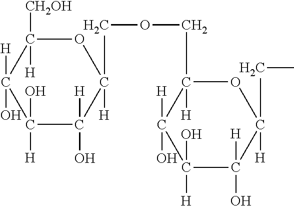

In the case of the di-, tri- or polyethers of ellagic acid, the R groups as defined above may be identical or different.

Ethers of this kind are described in U.S. Pat. No. 5,073,545. These ethers of ellagic acid are preferably selected from 3,4-di-O-methyl ellagic acid, 3,3′-4-tri-O-methyl ellagic acid and 3,3′-di-O-methyl ellagic acid.

The salt or salts of ellagic acid and/or of its ethers that can be used according to the invention are preferably selected from alkali metal or alkaline-earth metal salts, such as the sodium, potassium, calcium and magnesium salt, the ammonium salt and the salts of amines such as triethanolamine, monoethanolamine, arginine and lysine salts. Preferably, the salt or salts of ellagic acid and/or of its ethers that can be used according to the invention are selected from salts of alkali metals or alkaline-earth metals, more particularly from the sodium, potassium, calcium or magnesium salts.

Among all of the compounds (i) cited, preference is given to using ellagic acid or a salt thereof.

The composition according to the invention comprises preferably from 0.01% to 10% by weight, in particular from 0.1% to 5% by weight, and more preferably from 0.2% to 2% by weight of compound(s) (i), relative to the total weight of the composition.

The composition according to the invention comprises one or more compounds (ii) selected from:

1) salts of pyrithione. Pyrithione is the compound 1-hydroxy-2(1H)-pyridinethione or 2-pyridinethiol-1-oxide; the salts are, in particular, the monovalent metal salts and divalent metal salts, such as the sodium, calcium, magnesium, barium, strontium, zinc, cadmium, tin and zirconium salts. The divalent metal salts and more particularly the zinc salt (zinc pyrithione) are particularly preferred.

2) 1-hydroxy-2-pyridone derivatives. These derivatives are preferably selected from compounds of formula (A1) or salts thereof:

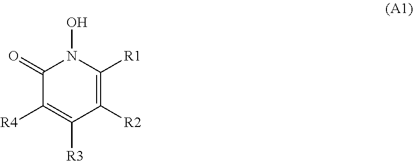

in which:

R1 denotes a hydrogen atom; a linear or branched alkyl group having from 1 to 17 carbon atoms; a cycloalkyl group having 5 to 8 carbon atoms; a cycloalkyl-alkyl group, the cycloalkyl group having 5 to 8 carbon atoms and the alkyl group having from 1 to 4 carbon atoms; an aryl or aralkyl group, the aryl group having from 6 to 30 carbon atoms and the alkyl group having from 1 to 4 carbon atoms; an aryl-alkenyl group, the aryl group having from 6 to 30 carbon atoms and the alkenyl group having from 2 to 4 carbon atoms; the cycloalkyl and aryl groups as defined above may be substituted by one or more alkyl groups having 1 to 4 carbon atoms or else one or more alkoxy groups having from 1 to 4 carbon atoms;

R2 denotes a hydrogen atom; an alkyl group having from 1 to 4 carbon atoms; an alkenyl group having from 2 to 4 carbon atoms; a halogen atom or a benzyl group;

R3 denotes a hydrogen atom, an alkyl group having from 1 to 4 carbon atoms or a phenyl group; and

R4 denotes a hydrogen atom; an alkyl group having from 1 to 4 carbon atoms; an alkenyl group having from 2 to 4 carbon atoms; a methoxymethyl group; a halogen atom or a benzyl group.

Among these compounds, those which are particularly preferred are 1-hydroxy-4-methyl-6-(2,4,4-trimethylpentyl)-2-(1H)-pyridone and 6-cyclohexyl-1-hydroxy-4-methyl-2-(1H)-pyridone.

The salts which can be used include the salts of lower (C₁-C₄) alkanolamines, such as ethanolamine and diethanolamine, amine or alkylamine salts, and also salts with inorganic cations, for instance ammonium salts and the salts of alkali metals or alkaline-earth metals.

Very particular preference is given to the monoethanolamine salt of 1-hydroxy-4-methyl-6-(2,4,4-trimethylpentyl)-2(1H)-pyridinone (or piroctone), more commonly referred to as piroctone olamine or octopirox.

3) selenium (poly)sulphides, especially selenium disulphide and the selenium polysulphides of formula SeₓS_{y} in which x and y are numbers such that x+y=8. Selenium disulphide takes the form of a powder with particles that in general have a size of less than 200 μm, preferably less than 25 μm.

All of these compounds (ii) are antidandruff agents.

The compound or compounds (ii) are preferably selected from zinc pyrithione, piroctone olamine and selenium disulphide.

The composition according to the invention comprises preferably from 0.01% to 10% by weight, in particular from 0.02% to 5% by weight, and more preferably from 0.04% to 2% by weight of compound(s) (ii), relative to the total weight of the composition.

The composition according to the invention is such that the weight ratio of the amount of compound(s) (i) to the amount of compound(s) (ii) is greater than or equal to 0.5.

The weight ratio of the amount of compound(s) (i) to the amount of compound(s) (ii) is preferably greater than or equal to 0.7, and more preferably greater than or equal to 1.

The weight ratio of the amount of compound(s) (i) to the amount of compound(s) (ii) is preferably less than or equal to 100, more preferably less than or equal to 50 and very preferably less than or equal to 20.

The composition according to the invention may also comprise one or more thickeners.

For the purposes of the present invention, a thickener is an agent which, when introduced at 1% by weight into an aqueous or aqueous-alcoholic solution containing 30% by weight of ethanol, and at a pH=7, produces a viscosity of at least 100 cPs, preferably at least 500 cPs, at 25° C. and at a shear rate of 1 s⁻¹. This viscosity may be measured using a cone/plate viscometer (Haake R600 Rheometer or the like).

The thickener or thickeners may be selected from sodium chloride, fatty acid amides obtained from C₁₀-C₃₀ carboxylic acid (monoisopropanolamide, diethanolamide or monoethanolamide of coconut acids, monoethanolamide of ethoxylated carboxylic alkyl ether acid), nonionic cellulosic thickeners (hydroxyethylcellulose, hydroxypropylcellulose, carboxymethylcellulose), guar gum and its nonionic derivatives (hydroxypropylguar), gums of microbial origin (xanthan gum, scleroglucan gum), crosslinked or non-crosslinked homopolymers and copolymers based on acrylic acid, methacrylic acid or acrylamidopropanesulphonic acid, and associative polymers as described below.

The associative polymer or polymers which can be used according to the invention are water-soluble polymers which in an aqueous medium are capable of reversible association with one another or with other molecules.

Their chemical structure comprises hydrophilic zones, and hydrophobic zones characterized by at least one fatty chain containing preferably from 10 to 30 carbon atoms.

The associative polymer or polymers which can be used according to the invention may be anionic, cationic, amphoteric or nonionic polymers, such as the polymers sold under the names Pemulen TR1 or TR2 by the company Goodrich (INCI: Acrylates/C10 30 Alkyl Acrylate Crosspolymer), Salcare SC90 by the company CIBA, Aculyn 22, 28, 33, 44 or 46 by the company Rohm & Haas, and Elfacos T210 and T212 by the company Akzo.

Among all of the thickeners mentioned, the thickener or thickeners are selected preferably from homopolymers and copolymers based on acrylic acid or methacrylic acid, preferably crosslinked polymers, and/or from fatty acid amides obtained from C₁₀-C₃₀ carboxylic acid.

The cosmetic composition comprises preferably from 0.1% to 20% by weight, and more preferably from 0.2% to 10% by weight, of thickener(s), relative to the total weight of the composition.

The composition according to the invention may further comprise one or more surfactants selected from anionic, nonionic, amphoteric or zwitterionic surfactants and mixtures thereof.

These surfactants are in particular mentioned in the CTFA (2004 edition) under the name “surfactant—cleansing agent”.

The term “anionic surfactant” refers to a surfactant which as ionic or ionisable groups contains only anionic groups. These anionic groups are selected preferably from the groups CO₂H, CO₂⁻, SO₃H, SO₃⁻, OSO₃H, OSO₃⁻, O₂PO₂H, O₂PO₂H⁻ and O₂PO₂²⁻.

The anionic surfactant or surfactants which may be used in the compositions of the invention are selected in particular from alkyl sulphates, alkyl ether sulphates, alkylamidoether sulphates, alkylaryl polyether sulphates, monoglyceride sulphates, alkyl sulphonates, alkylamidesulphonates, alkylarylsulphonates, alpha-olefin-sulphonates, paraffinsulphonates, alkyl sulphosuccinates, alkyl ether sulphosuccinates, alkylamidesulphosuccinates, alkyl sulphoacetates, acylsarcosinates, acylglutamates, alkylsulphosuccinamates, acylisethionates and N-acyltaurates, salts of alkyl monoesters and polyglycoside-polycarboxylic acids, acyllactylates, salts of D galactoside-uronic acids, salts of alkyl ether-carboxylic acids, salts of alkyl aryl ether-carboxylic acids, and salts of alkyl amidoether-carboxylic acids; or the non-salified forms of all of these compounds, with the alkyl and acyl groups of all of these compounds containing from 6 to 24 carbon atoms and the aryl group denoting a phenyl group.

Some of these compounds may be ethoxylated and if so contain preferably from 1 to 50 ethylene oxide units.

The salts of C₆₋₂₄ alkyl monoesters and polyglycoside-polycarboxylic acids may be selected from C₆₋₂₄ alkyl polyglycoside-citrates, C₆₋₂₄ alkyl polyglycoside-tartrates and C₆₋₂₄ alkyl polyglycoside-sulphosuccinates.

When the anionic surfactant(s) are in salt form, it (they) can be selected from alkali metal salts, such as the sodium or potassium salt, and preferably the sodium salt, ammonium salts, amine salts, and in particular aminoalcohol salts, and alkaline-earth metal salts such as the magnesium salt.

Examples of aminoalcohol salts include more particularly mono-, di- and triethanolamine salts, mono-, di- or tri-isopropanol [symbol] amine salts, 2-amino-2-methyl-1-propanol salts, 2-amino-2-methyl-1,3-propanediol salts and tris(hydroxymethyl)aminomethane salts.

The alkali metal or alkaline-earth metal salts and in particular the sodium or magnesium salts are preferably used.

Preference is given to using (C₆₋₂₄)alkyl sulphates and (C₆₋₂₄)alkyl ether sulphates, which are optionally ethoxylated, containing from 2 to 50 units of ethylene oxide, and mixtures thereof, especially in the form of alkali metal salts or alkaline earth metal salts, ammonium salts or aminoalcohol salts. More preferably, the anionic surfactant or surfactants are selected from (C₁₀₋₂₀) alkyl ether sulphates, and especially sodium lauryl ether sulphate containing 2.2 mol of ethylene oxide.

When they are present, the amount of the anionic surfactant or surfactants varies preferably from 0.1% to 50% by weight, more preferably from 4% to 30% by weight, relative to the total weight of the composition.

Examples of nonionic surfactants that may be used in the cosmetic composition used according to the invention are described, for example, in the “Handbook of Surfactants” by M. R. PORTER, published by Blackie & Son (Glasgow and London), 1991, pp. 116-178. They are selected more particularly from alcohols, alpha-diols, (C₁₋₂₀)alkyl-phenols or polyethoxylated, polypropoxylated and/or polyglycerolated fatty acids having a fatty chain containing, for example, from 8 to 18 carbon atoms, it being possible for the number of ethylene oxide and/or propylene oxide groups to be in particular from 2 to 50 and for the number of glycerol groups to be in particular from 2 to 30.

Mention may also be made of copolymers of ethylene oxide and propylene oxide, optionally ethoxylated esters of fatty acids and sorbitan, esters of fatty acids and sucrose, polyoxyalkylenated fatty acid esters, optionally alkoxylated alkylpolyglycosides, alkylglucoside esters, derivatives of N-alkylglucamine and of N-acylmethyl-glucamine, aldobionamides and amine oxides.

Unless otherwise mentioned, the term “fatty” compound (for example a fatty acid) denotes a compound comprising, in its main chain, at least one saturated or unsaturated alkyl chain containing at least 8 carbon atoms, preferably from 8 to 30 carbon atoms, and even better still from 10 to 22 carbon atoms.

When they are present, the amount of the nonionic surfactant or surfactants varies preferably from 0.01% to 20% by weight, more preferably from 0.2% to 10% by weight, relative to the total weight of the composition.

The amphoteric or zwitterionic surfactant(s) that can be used in the present invention can in particular be derivatives of secondary or tertiary aliphatic amines, which are optionally quaternized, in which the aliphatic group is a linear or branched chain containing from 8 to 22 carbon atoms, the said amine derivatives containing at least one anionic group such as, for example, a carboxylate, sulphonate, sulphate, phosphate or phosphonate group. Mentioned in particular are (C₈₋₂₀) alkyl betaines, sulphobetaines, (C₈₋₂₀ alkyl)amido (C₂₋₈ alkyl) betaines or (C₈₋₂₀ alkyl) amido (C₂₋₈ alkyl) sulphobetaines.

Among the derivatives of secondary or tertiary aliphatic amines which are optionally quaternized and may be used as defined above, mention may also be made of the compounds with respective structures (A2) and (A3) below:

Rₐ₋CONHCH₂CH2-N⁺(R_{b})(R_{c})(CH₂COO⁻) (A2)

in which:

Rₐ represents a C₁₀-C₃₀ alkyl or alkenyl group derived from an acid Rₐ COOH which is preferably present in hydrolysed coconut oil, or a heptyl, nonyl or undecyl group,

R_{b} represents a beta-hydroxyethyl group, and

R_{c} represents a carboxymethyl group; and

R_{a′}—CONHCH₂CH₂—N(B)(B′) (A3)

in which:

B represents —CH₂CH₂OX′,

B′ represents —(CH₂)_{z}—Y′, with z=1 or 2,

X′ represents the group —CH₂—COOH, CH₂—COOZ′, —CH₂CH₂—COOH, —CH₂CH₂—COOZ′, or a hydrogen atom,

Y′ represents —COOH, —COOZ′, the group —CH₂—CHOH—SO₃H or —CH₂—CHOH—SO₃Z′,

Z′ represents an ion derived from an alkali or alkaline-earth metal, such as sodium, potassium or magnesium; an ammonium ion; or an ion derived from an organic amine and in particular from an aminoalcohol, such as mono-, di- and triethanolamine, mono-, di- or triisopropanolamine, 2-amino-2-methyl-1-propanol, 2-amino-2-methyl-1,3-propanediol and tris(hydroxymethyl)aminomethane.

Ra′ represents a C₁₀-C₃₀ alkyl or alkenyl group of an acid Rₐ′COOH which is preferably present in coconut oil or in hydrolysed linseed oil, or an alkyl group, especially a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Among the abovementioned amphoteric or zwitterionic surfactants it is preferred to use (C₈₋₂₀ alkyl) betaines such as cocoylbetaine, (C₈₋₂₀ alkyl) amido (C₂₋₈ alkyl) betaines such as cocoylamidopropylbetaine, and mixtures thereof. More preferably, the amphoteric or zwitterionic surfactant(s) is (are) selected from cocoylamidopropylbetaine and cocoylbetaine.

When they are present, the amount of the amphoteric or zwitterionic surfactant or surfactants is preferably in the range from 0.01% to 20% by weight, more preferably from 0.5% to 10% by weight, relative to the total weight of the composition.

The composition according to the invention is generally used in topical application.

In particular, the composition according to the invention may be in any of the presentation forms normally used for topical application.

The cosmetic composition used according to the invention may be a rinse-out or leave-in composition. More particularly it may be a shampoo, a cream, a mousse (aerosol or not), a paste, a gel, an emulsion, etc. Preferably, the cosmetic composition is a shampoo or a gel.

In a first preferred embodiment, the composition according to the invention is in the form of a gel. In this case, it comprises at least one thickener that is preferably present in an amount sufficient to give a gel.

A “gel” or “gelled composition” is a composition having a viscosity of from 100 cPs to 500 000 cPs, preferably from 200 cPs to 100 000 cPs, at ambient temperature (25° C.) and under atmospheric pressure (1 bar) at a shear rate of 1 s⁻¹. This viscosity may be measured using a cone/plate viscometer (Haake R600 Rheometer or the like).

This particular form allows the composition to remain localized at the point of application. Thus, the composition does not run, thereby reducing the risks of the composition coming into contact with the eyes.

The thickener(s) may be selected from those mentioned hereinabove.

In a second preferred embodiment, the composition according to the invention is in the form of a shampoo. In this case, the composition comprises one or more surfactants that are preferably present in an amount sufficient to wash the hair.

The surfactant or surfactants may be selected from those defined above.

The total amount of anionic, nonionic, and/or amphoteric or zwitterionic surfactants is then preferably at least 3% by weight, relative to the total weight of the composition. Even more preferably, the composition according to the invention has a total content of anionic, nonionic, and/or amphoteric or zwitterionic surfactants ranging from 4% to 50% by weight, even better still from 4% to 20% by weight, relative to the total weight of the composition.

The composition according to the invention generally comprises a cosmetically acceptable medium. The medium is composed of water and optionally one or more cosmetically acceptable organic solvents.

The organic solvent or solvents may be selected from C₁-C₄ lower alcohols, such as ethanol, isopropanol, tert-butanol or n-butanol; polyols such as glycerol, propylene glycol, hexylene glycol (or 2 methyl-2,4-pentanediol) and polyethylene glycols; polyol ethers such as dipropylene glycol monomethyl ether; and mixtures thereof.

The cosmetic composition used according to the invention preferably contains an amount of organic solvents of from 0.05% to 60%, preferably from 0.5% to 50%, and more preferably from 1% to 40% by weight, relative to the total weight of the composition.

The composition according to the invention may also comprise one or more conditioning agents.

According to the present invention, the term “conditioning agent” denotes any compound that can improve the cosmetic properties of the hair, in particular the softness, disentangling, feel and static electricity.

The conditioning agent is preferably selected from the group consisting of cationic polymers, cationic surfactants, silicones such as organosiloxanes, linear or branched C₈-C₃₀ hydrocarbons, linear or branched C₈-C₃₀ fatty alcohols, esters of C₈-C₃₀ fatty acid and C₁-C₃₀ alcohol, and especially esters of C₈-C₃₀ fatty acid and C₈-C₃₀ fatty alcohol, esters of C₁-C₇ acid or diacid and C₈-C₃₀ fatty alcohol, ceramides or ceramide analogues, and the mixtures of these compounds.

The term “cationic polymer” means a polymer that is positively charged when it is contained in the composition according to the invention. This polymer may carry one or more positive permanent charges or may contain one or more cationizable functions within the composition according to the invention.

The cationic polymer(s) that may be used as conditioning agents according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

When the conditioning agent is a cationic polymer, it is preferably selected from those that contain units comprising primary, secondary, tertiary and/or quaternary amine groups that may either form part of the main polymer chain, or may be carried by a side substituent directly attached thereto.

Among the cationic polymers that may be mentioned more particularly are polymers of the polyamine, polyaminoamide and polyquaternary ammonium type. These are known products. They are described, for example, in French patents 2 505 348 and 2 542 997.

Among these polymers, mention may be made of:

(1) homopolymers or copolymers derived from acrylic or methacrylic esters or amides and comprising at least one of the units of the following formulae:

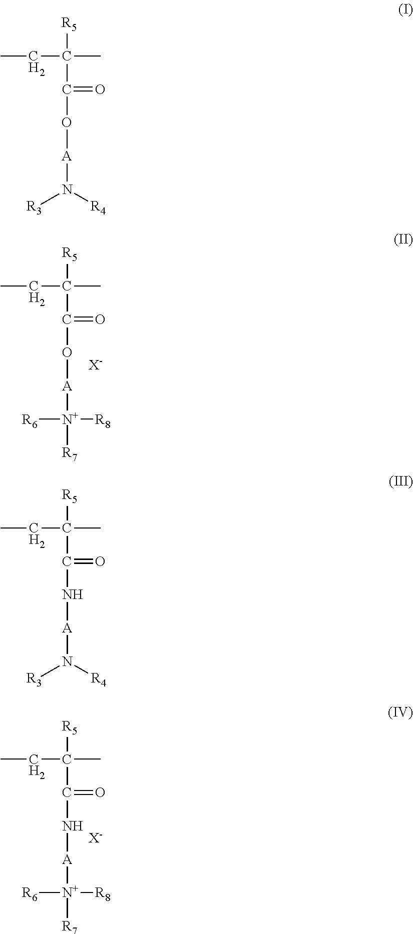

in which:

R₃ and R₄, which are identical or different, represent a hydrogen atom or an alkyl group having from 1 to 6 carbon atoms, and preferably methyl or ethyl;

R₅, identical or different at each occurrence, denotes a hydrogen atom or a group CH3;

A, which are identical or different, represent a linear or branched alkyl group having from 1 to 6 carbon atoms, preferably 2 or 3 carbon atoms, or a hydroxyalkyl group having from 1 to 4 carbon atoms;

R₆, R₇ and R₈, which are identical or different, represent an alkyl group having from 1 to 18 carbon atoms or a benzyl group, and preferably an alkyl group having from 1 to 6 carbon atoms;

X⁻ denotes an anion derived from an organic or inorganic acid, such as a methosulphate anion, or a halide such as chloride or bromide.

The copolymers of the family (1) may further contain one or more units deriving from comonomers which may be selected from the family of acrylamides, methacrylamides, diacetone acrylamides, acrylamides and methacrylamides substituted on the nitrogen by lower alkyls (C₁-C₄), acrylic acids or methacrylic acids or esters thereof, vinyl lactams such as vinylpyrrolidone or vinylcaprolactam, and vinyl esters.

Accordingly, among these copolymers of the family (1), mention may be made of the following:
copolymers of acrylamide and of dimethylaminoethyl methacrylate quaternized with dimethyl sulphate or with a dimethyl halide, such as that sold under the name Hercofloc by the company Hercules,
copolymers of acrylamide and of methacryloyloxyethyltri-methylammonium chloride, described, for example, in patent application EP-A-080976 and sold under the name Bina Quat P 100 by the company Ciba Geigy,
the copolymer of acrylamide and of methacryloyloxyethyltri-methylammonium methosulphate sold under the name Reten by the company Hercules,
quaternized or non-quaternized vinylpyrrolidone/dialkyl-aminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinyl-pyrrolidone terpolymers, such as the product sold under the name Gaffix VC 713 by the company ISP,
the vinylpyrrolidone/methacrylamidopropyldimethylamine copolymers sold in particular under the name Styleze CC 10 by ISP,
quaternized vinylpyrrolidone/dimethylaminopropyl [symbol] methacryl [symbol] amide copolymers, such as the product sold under the name Gafquat HS 100 by the company ISP, and
the crosslinked polymers of methacryloyloxy(C₁-C₄)alkyl tri(C₁-C₄) alkylammonium salts, such as the polymers obtained by homopolymerization of quaternized dimethylaminoethyl methacrylate with methyl chloride, or by copolymerization of acrylamide with dimethylaminoethyl methacrylate quaternized with methyl chloride, the homo- or copolymerization being followed by crosslinking with an olefinically unsaturated compound, more particularly methylenebis [symbol] acrylamide. Use may be made more particularly of a crosslinked acrylamide/methacryloyloxyethyltrimethylammonium chloride copolymer (20/80 by weight) in the form of a dispersion containing 50% by weight of the said copolymer in mineral oil. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.

(2) Polymers composed of piperazinyl units and of straight-chain or branched-chain divalent alkylene or hydroxyalkylene groups, optionally interrupted by oxygen, sulphur or nitrogen atoms or by aromatic or heterocyclic rings, and also the oxidation and/or quaternization products of these polymers. Polymers of this kind are described in particular in French patents 2 162 025 and 2 280 361;

(3) Water-soluble polyaminoamides, prepared in particular by polycondensation of an acid compound with a polyamine; these polyaminoamides may be crosslinked with an epihalohydrin, a diepoxide, a dianhydride, an unsaturated dianhydride, a bis-unsaturated derivative, a bis-halohydrin, a bis-azetidinium, a bis-haloacyldiamine or a bis-alkyl halide or else by an oligomer resulting from the reaction of a bifunctional compound which is reactive towards a bis-halohydrin, a bis-azetidinium, a bis-haloacyldiamine, a bis-alkyl halide, an epihalohydrin, a diepoxide or a bis-unsaturated derivative; the crosslinking agent being used in proportions of from 0.025 to 0.35 mol per amine group of the polyaminoamide; these polyaminoamides may be alkylated, or quaternized if they contain one or more tertiary amine functions. Polymers of this kind are described in particular in French patents 2 252 840 and 2 368 508;

(4) Polyaminoamide derivatives resulting from the condensation of polyalkylene polyamines with polycarboxylic acids, followed by alkylation with difunctional agents. Examples include adipic acid-dialkylaminohydroxyalkyldialkylenetriamine polymers in which the alkyl groups contain from 1 to 4 carbon atoms and are preferably each a methyl, ethyl or propyl group, and the alkylene groups contain from 1 to 4 carbon atoms and preferably denote the ethylene group. Polymers of this kind are described in particular in French patent 1 583 363.

Among these derivatives, mention may be made more particularly of the adipic acid/dimethylaminohydroxy-propyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyaminoamide resulting therefrom is reacted with epichlorohydrin in a molar ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(6) Alkyldiallylamine or dialkyldiallylammonium cyclopolymers, such as the homopolymers or copolymers comprising as their principal chain constituent units conforming to the formulae (V) or (VI):

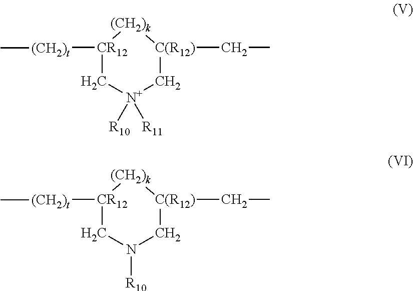

in which formulae: k and t are 0 or 1, the sum k+t being 1; R₁₂ denotes a hydrogen atom or a methyl group; R₁₀ and R₁₁ independently of one another denote an alkyl group having from 1 to 6 carbon atoms, a hydroxyalkyl group in which the alkyl group has preferably 1 to 5 carbon atoms, a lower (C₁-C₄) amidoalkyl group, or else R₁₀ and R₁₁ may, together with the nitrogen atom to which they are attached, denote heterocyclic groups, such as piperidyl or morpholinyl; Y⁻ is an anion such as bromide, chloride, acetate, borate, citrate, tartrate, bisulphate, bisulphite, sulphate or phosphate. These polymers are described in particular in French patent 2 080 759 and in its certificate of addition 2 190 406.

R₁₀ and R₁₁ independently of one another denote preferably an alkyl group having from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) The quaternary diammonium polymer containing repeating units conforming to the formula (VII):

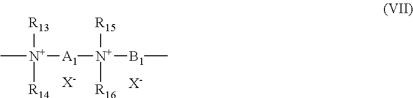

in which formula (VII):

R₁₃, R₁₄, R₁₅ and R₁₆, which are identical or different, represent aliphatic, alicyclic or arylaliphatic groups containing from 1 to 20 carbon atoms, or lower (C₁-C₄) hydroxyalkylaliphatic groups, or else R₁₃, R₁₄, R₁₅ and R₁₆, together or separately, with the nitrogen atoms to which they are attached, form heterocycles optionally containing a second heteroatom other than the nitrogen, or else R₁₃, R₁₄, R₁₅ and R₁₆ represent a linear or branched C₁-C₆ alkyl group substituted by a nitrile, ester, acyl or amide group or by a group —CO—O—R₁₇-D or —CO—NH—R₁₇-D in which R₁₇ is an alkylene having from 1 to 10 carbon atoms and D is a quaternary ammonium group;

A₁ and B₁ represent polymethylene groups containing from 2 to 20 carbon atoms, which may be linear or branched and saturated or unsaturated and may contain, joined to or intercalated in the main chain, one or more aromatic rings, or one or more oxygen or sulphur atoms or sulphoxide, sulphone, disulphide, amino, alkylamino, hydroxyl, quaternary ammonium, ureido, amide or ester groups, and

X⁻ denotes an anion derived from an organic or inorganic acid;

A₁, R₁₃ and R₁₅ may, with the two nitrogen atoms to which they are attached, form a piperazine ring; moreover, if A₁ denotes a linear or branched, saturated or unsaturated alkylene or hydroxyalkylene group, B₁ may also denote a group

—(CH₂)ₙ—CO-D-OC—(CH₂)ₚ—

in which:

n and p are integers ranging from 2 to 20 approximately,

D denotes:

a) a glycol residue of formula: —O—Z—O—, where Z denotes a linear or branched hydrocarbon group or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a single, defined degree of polymerization, or any number from 1 to 4, representing an average degree of polymerization;

b) a bis-secondary diamine residue such as a piperazine derivative;

c) a bis-primary diamine residue of formula: —NH—Y—NH—, in which Y denotes a linear or branched hydrocarbon group, or else the divalent group —CH₂—CH₂—S—S—CH₂—CH₂—;

d) a ureylene group of formula: —NH—CO—NH—;

X⁻ is preferably an anion such as chloride or bromide.

These polymers have a number-average molecular mass of generally between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may more particularly be made of polymers which are composed of repeating units corresponding to the formula (VIII):

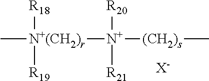

in which: R₁₈, R₁₉, R₂₀ and R₂₁, which are identical or different, denote an alkyl or hydroxyalkyl group having from 1 to 4 carbon atoms approximately, r and s are integers from 2 to 20 approximately, and X— is an anion derived from an organic or inorganic acid.

One particularly preferred compound of formula (VIII) is that for which R₁₈, R₁₉, R₂₀ and R₂₁ represent a methyl group and r=3, s=6 and X=Cl, which is called Hexadimethrine chloride according to the INCI nomenclature (CTFA).

(8) Polyquaternary ammonium polymers composed of units of formula (IX):

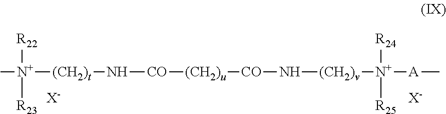

in which formula:

R₂₂, R₂₃, R₂₄ and R₂₅, which are identical or different, represent a hydrogen atom or a methyl, ethyl, propyl, β-hydroxyethyl, β-hydroxypropyl or —CH₂CH₂(OCH₂CH₂)pOH group, where p is 0 or an integer between 1 and 6, with the proviso that R₂₂, R₂₃, R₂₄ and R₂₅ do not simultaneously represent a hydrogen atom,

t and u, which are identical or different, are integers between 1 and 6,

v is equal to 0 or to an integer between 1 and 34,

X⁻ denotes an anion such as a halide,

A denotes a group of a dihalide or represents preferably —CH₂ CH₂—O—CH₂—CH₂—.

Compounds of this kind are described in particular in patent application EP-A-122 324.

Mention among these may be made, for example, of the products Mirapol® A 15, Mirapol® AD1, Mirapol® AZ1 and Mirapol® 175, which are sold by the company Miranol.

(9) Quaternary polymers of vinylpyrrolidone and vinylimidazole, such as, for example, the products sold under the names Luviquat® FC 905, FC 550 and FC 370 by the company BASF.

(10) Cationic polysaccharides, more particularly cationic celluloses and derivatives of cationic celluloses, and cationic galactomannan gums.

The cationic polysaccharides include more particularly cellulose ether derivatives containing quaternary ammonium groups, cationic cellulose copolymers or cellulose derivatives grafted with a water-soluble quaternary ammonium monomer, and cationic galactomannan gums.

The cellulose ether derivatives comprising quaternary ammonium groups described in French patent 1 492 597. These polymers are also defined in the CTFA dictionary as quaternary ammoniums of hydroxyethylcellulose that have reacted with an epoxide substituted by a trimethylammonium group.

The cationic cellulose copolymers or the cellulose derivatives grafted with a water-soluble quaternary ammonium monomer are described especially in patent U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamidopropyl-trimethylammonium or dimethyldiallylammonium salt.

Cationic galactomannan gums are described more particularly in U.S. Pat. Nos. 3,589,578 and 4,031,307, especially the guar gums containing cationic trialkylammonium groups. Use is made, for example, of guar gums modified with a salt (e.g. chloride) of 2, 3-epoxypropyltrimethylammonium.

Other cationic polymers which can be used in the context of the invention are cationic proteins or cationic protein hydrolysates, polyalkyleneimines, more particularly polyethyleneimines, polymers containing vinylpyridine or vinylpyridinium units, condensates of polyamines and epichlorhydrin, quaternary polyureylenes, and derivatives of chitin.

The cationic proteins or protein hydrolysates are, in particular, chemically modified polypeptides bearing quaternary ammonium groups at the end of the chain, or grafted thereon. Their molecular mass may vary, for example, from 1500 to 10 000 and in particular from 2000 to 5000 approximately. Among these compounds, mention may be made especially of:
collagen hydrolysates carrying triethylammonium groups, such as the products sold under the name Quat-Pro E by the company Maybrook and referred to in the CTFA dictionary as Triethonium Hydrolyzed Collagen Ethosulfate;
collagen hydrolysates carrying trimethylammonium chloride and trimethylstearylammonium chloride groups, which are sold under the name Quat-Pro S by the company Maybrook and are referred to in the CTFA dictionary as Steartrimonium Hydrolyzed Collagen;
animal protein hydrolysates which carry trimethylbenzyl [symbol] ammonium groups, such as the products sold under the name Crotein BTA by the company Croda and referred to in the CTFA dictionary as Benzyltrimonium hydrolyzed animal protein;
protein hydrolysates carrying quaternary ammonium groups on the polypeptide chain, the said ammonium groups containing at least one alkyl group having from 1 to 18 carbon atoms.

Among these protein hydrolysates, mention may be made, inter alia, of:
Croquat L, in which the quaternary ammonium groups contain a C₁₂ alkyl group;
Croquat M, in which the quaternary ammonium groups contain C₁₀-C₁₈ alkyl groups;
Croquat S, in which the quaternary ammonium groups contain a C₁₈ alkyl group;
Crotein Q, in which the quaternary ammonium groups contain at least one alkyl group having from 1 to 18 carbon atoms.

These various products are sold by the company Croda.

Other quaternized proteins or hydrolysates are, for example, those corresponding to the formula (X):

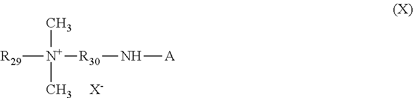

in which X⁻ is an anion of an organic or inorganic acid, A denotes a protein residue derived from collagen protein hydrolysates, R²⁹ denotes a lipophilic group containing up to 30 carbon atoms, R³⁰ represents an alkylene group having 1 to 6 carbon atoms. Examples include the products sold by the company Inolex, under the name Lexein QX 3000, which in the CTFA dictionary is called Cocotrimonium Collagen Hydrolysate.

Mention may also be made of quaternized plant proteins, such as wheat, corn or soya proteins: quaternized wheat proteins include those sold by the company Croda under the name Hydrotriticum WQ or QM, which in the CTFA dictionary are called Cocodimonium Hydrolysed wheat protein, or Hydrotriticum QL, which in the CTFA dictionary is called Laurdimonium hydrolysed wheat protein, or else Hydrotriticum QS, which in the CTFA dictionary is called Steardimonium hydrolysed wheat protein.

Among all the cationic polymers that may be used in the context of the present invention, it is preferred to use cationic cyclopolymers as defined above, in particular the dimethyldiallylammonium chloride homopolymers or copolymers sold under the names Merquat 100, Merquat 550 and Merquat S by the company Nalco, and quaternary vinylpyrrolidone and vinylimidazole polymers, cationic polysaccharides and mixtures thereof.

The conditioning agent(s) that can be used according to the invention may be selected from cationic surfactants.

A “cationic surfactant” means a surfactant which is positively charged when it is present in the composition according to the invention. This surfactant may carry one or more positive permanent charges or may contain one or more cationizable functions within the composition according to the invention.

The cationic surfactant(s) that may be used as conditioning agents according to the present invention are preferably selected from primary, secondary or tertiary fatty amines, optionally polyoxyalkylenated, or salts thereof, and quaternary ammonium salts, and mixtures thereof.

The fatty amines generally comprise at least one C⁸-C³⁰ hydrocarbon chain. Among the fatty amines that may be used according to the invention, examples that may be mentioned include stearylamidopropyldimethylamine and distearylamine.

Examples of quaternary ammonium salts that may especially be mentioned include:
those corresponding to the general formula (XI) below:

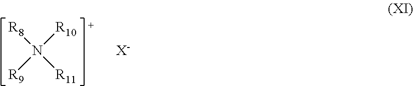

in which the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ denoting a group containing from 8 to 30 carbon atoms, preferably from 12 to 24 carbon atoms. The aliphatic groups can comprise heteroatoms such as, in particular, oxygen, nitrogen, sulphur and halogens. The aliphatic groups are for example selected from C₁₋₃₀ alkyl, C₁₋₃₀ alkoxy, polyoxyalkylene (C₂-C₆), C₁₋₃₀ alkylamide, (C₁₂-C₂₂)alkylamido-(C₂ C₆)alkyl, (C₁₂-C₂₂)alkyl acetate and C₁₋₃₀ hydroxyalkyl groups; X— is an anion selected from the group consisting of halides, phosphates, acetates, lactates, (C₁-C₄)alkyl sulphates, (C₁-C₄)alkyl sulphonates and (C₁-C₄)alkyl-aryl sulphonates.

Among the quaternary ammonium salts of formula (XI), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethyl [symbol] ammonium, cetyltrimethylammonium or benzyldimethylstearyl [symbol] ammonium salts, or, on the other hand, the palmitylamidopropyl [symbol] trimethylammonium salt, the stearamidopropyltrimethylammonium salt, the stearamidopropyldimethylcetearylammonium salt, or the stearamidopropyldimethyl(myristyl acetate)ammonium salt sold under the name Ceraphyl® 70 by the company Van Dyk. It is particularly preferred to use the chloride salts of these compounds;
quaternary ammonium salts of imidazoline, such as, for example, those of formula (XII) below:

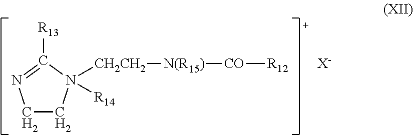

in which R₁₂ represents an alkyl or alkenyl group containing from 8 to 30 carbon atoms, derived for example from tallow fatty acids, R₁₃ represents a hydrogen atom, a C₁-C₄ alkyl group or an alkyl or alkenyl group containing from 8 to 30 carbon atoms, R₁₄ represents a C₁-C₄ alkyl group, R₁₅ represents a hydrogen atom or a C₁-C₄ alkyl group, X— is an anion selected from the group consisting of halides, phosphates, acetates, lactates, alkyl sulphates, alkylsulphonates or alkylarylsulphonates in which the alkyl and aryl groups each preferably comprise from 1 to 20 carbon atoms and from 6 to 30 carbon atoms. R₁₂ and R₁₃ preferably denote a mixture of alkyl or alkenyl groups containing from 12 to 21 carbon atoms, derived for example from tallow fatty acids, R₁₄ preferably denotes a methyl group, and R₁₅ preferably denotes a hydrogen atom. A product of this kind is sold for example under the name Rewoquat® W 75 by the company Rewo;
quaternary diammonium or triammonium salts, particularly of formula (XIII):

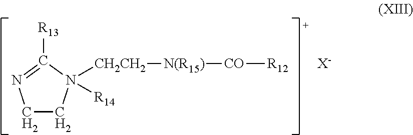

in which R₁₂ represents an alkyl or alkenyl group containing from 8 to 30 carbon atoms, derived for example from tallow fatty acids, R₁₃ represents a hydrogen atom, a C₁-C₄ alkyl group or an alkyl or alkenyl group containing from 8 to 30 carbon atoms, R₁₄ represents a C₁-C₄ alkyl group, R₁₅ represents a hydrogen atom or a C₁-C₄ alkyl group, X— is an anion selected from the group consisting of halides, phosphates, acetates, lactates, alkyl sulphates, alkylsulphonates or alkylarylsulphonates in which the alkyl and aryl groups each preferably comprise from 1 to 20 carbon atoms and from 6 to 30 carbon atoms. R₁₂ and R₁₃ preferably denote a mixture of alkyl or alkenyl groups containing from 12 to 21 carbon atoms, derived for example from tallow fatty acids, R₁₄ preferably denotes a methyl group, and R₁₅ preferably denotes a hydrogen atom. A product of this kind is sold for example under the name Rewoquat® W 75 by the company Rewo;
quaternary ammonium salts containing at least one ester function, such as those of formula (XIV) below:

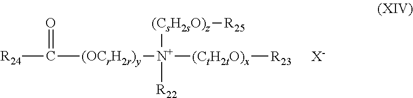

in which:

R₂₂ is selected from C₁-C₆ alkyl groups and C₁-C₆ hydroxyalkyl or dihydroxyalkyl groups;
R₂₃ is selected from:

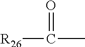

the group
groups R₂₇, which are linear or branched, saturated or unsaturated C₁-C₂₂ hydrocarbon groups,
a hydrogen atom,
R₂₅ is selected from:

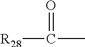

the group
groups R₂₉, which are linear or branched, saturated or unsaturated C₁-C₆ hydrocarbon groups,
a hydrogen atom,

R₂₄, R₂₆ and R₂₈, which are identical or different, are selected from linear or branched, saturated or unsaturated C₇-C₂₁ hydrocarbon groups;

r, s and t, which are identical or different, are integers from 2 to 6;

y is an integer ranging from 1 to 10;

x and z, which are identical or different, are integers ranging from 0 to 10;

X⁻ is a simple or complex, organic or inorganic anion;

with the proviso that the sum x+y+z is from 1 to 15, that when x is 0, R₂₃ denotes R₂₇ and that when z is 0, R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is an R₂₇ hydrocarbon group, it may be long and may have from 12 to 22 carbon atoms, or may be short and may have from 1 to 3 carbon atoms.

When R₂₅ is an R₂₉ hydrocarbon group, it has preferably 1 to 3 carbon atoms.

Advantageously, R₂₄, R₂₆ and R₂₈, which are identical or different, are selected from linear or branched, saturated or unsaturated C₁₁-C₂₁ hydrocarbon groups, and more particularly from linear or branched, saturated or unsaturated C₁₁-C₂₁ alkyl and alkenyl groups.

Preferably, x and z, which are identical or different, are 0 or 1.

Advantageously, y is equal to 1.

Preferably, r, s and t, which are identical or different, are 2 or 3, and even more particularly are equal to 2.

The anion X⁻ is preferably a halide (chloride, bromide or iodide) or an alkyl sulphate, more particularly methyl sulphate. It is possible, however, to use methanesulphonate, phosphate, nitrate or tosylate, an anion derived from organic acid, such as acetate or lactate, or any other anion which is compatible with ester-functional ammonium.

The anion X⁻ is more particularly still chloride or methyl sulphate.

More particularly used in the composition according to the invention are the ammonium salts of formula (XIV) in which:

R₂₂ denotes a methyl or ethyl group,

x and y are equal to 1;

z is equal to 0 or 1;

r, s and t are equal to 2;
R₂₃ is selected from:

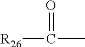

the group
methyl, ethyl or C₁₄-C₂₂ hydrocarbon groups,
a hydrogen atom;
R₂₅ is selected from:

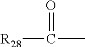

the group
a hydrogen atom;

R₂₄, R₂₆ and R₂₈, which are identical or different, are selected from linear or branched, saturated or unsaturated C₁₃-C₁₇ hydrocarbon groups, and preferably from linear or branched, saturated or unsaturated C₁₃-C₁₇ alkyl and alkenyl groups.

The hydrocarbon groups are advantageously linear.

Mention may be made, for example, of the compounds of formula (XIV) such as the diacyloxyethyldimethylammonium, diacyloxyethylhydroxyethylmethylammonium, monoacyloxyethyl-dihydroxyethylmethylammonium, triacyloxyethylmethylammonium and monoacyloxyethylhydroxyethyldimethylammonium salts (chloride or methyl sulphate in particular), and mixtures thereof. The acyl groups have preferably 14 to 18 carbon atoms and originate more particularly from a plant oil such as palm oil or sunflower oil. When the compound contains two or more acyl groups, these groups may be identical or different.

These products are obtained, for example, by direct esterification of triethanolamine, of triisopropanolamine, of alkyldiethanolamine or of alkyldiisopropanolamine, which are optionally alkoxylated, with C₁₀-C₃₀ fatty acids or with mixtures of C₁₀-C₃₀ fatty acids of plant or animal orgin, or by transesterification of their methyl esters. This esterification is followed by a quaternization by means of an alkylating agent such as an alkyl (preferably methyl or ethyl) halide, a dialkyl (preferably methyl or ethyl) sulphate, methyl methanesulphonate, methyl para-toluene-sulphonate, glycol chlorohydrin or glycerol chlorohydrin.

Such compounds are sold, for example, under the names Dehyquart® by the company Henkel, Stepanquat® by the company Stepan, Noxamium® by the company CECA and Rewoquat® WE 18 by the company Rewo-Witco.

The composition according to the invention may contain, for example, a mixture of quaternary ammonium salts of mono-, di- and triesters with a weight majority of diester salts.

Mixtures of ammonium salts that can be used include, for example, the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl groups having from 14 to 18 carbon atoms and originating from palm oil, which is optionally partially hydrogenated.

It is also possible to use the ammonium salts containing at least one ester function that are described in U.S. Pat. No. 4,874,554 and U.S. Pat. No. 4,137,180.

The particularly preferred cationic surfactant or surfactants that can be used according to the invention are selected from compounds of formula (XI) or of formula (XIV), methyl (C₉-C₁₉)alkyl-(C₁₀-C₂₀) alkylamidoethylimidazolium salts, and stearamidopropyldi [symbol] methylamine.

Among all of the cationic surfactants that may be present in the composition according to the invention, it is preferred to select cetyltrimethylammonium, behenyltrimethylammonium, di(palmitoyl-oxyethyl)hydroxyethylmethylammonium, di(stearoyloxyethyl)hydroxy-ethylmethylammonium, methyl (C₉-C₁₉)alkyl(C₁₀-C₂₀)alkylamido-ethylimidazolium salts, stearamidopropyltrimethylammonium salt, stearamidopropyldimethylamine, stearamidopropyldimethylcetearyl [symbol] ammonium salt, and mixtures thereof.

Among the silicones that can be used as conditioning agents in accordance with the present invention, mention may be made, without limitation, of the following:

I. Volatile Silicones:

These silicones have a boiling point of between 60° C. and 260° C. Among the silicones of this type that are mentioned are:

(a) cyclic silicones containing from 3 to 7 silicon atoms, preferably 4 to 5.

These are, for example, the octamethylcyclotetrasiloxane sold under the name Volatile Silicone 7207® by the company Union Carbide, or Silbione 70045 V2® by the company Rhone Poulenc, the decamethylcyclopentasiloxane sold under the name Volatile Silicone 7158® by the company Union Carbide or Silbione 70045 V5® by the company Rhone Poulenc, and mixtures thereof. Mention is also made of cyclopolymers of the dimethylsiloxane/methylalkylsiloxane type, such as Volatile Silicone FZ 3109® sold by the company Union Carbide, which is a dimethylsiloxane/methyloctylsiloxane cyclopolymer;

(b) linear volatile silicones having 2 to 9 silicon atoms and possessing a viscosity of less than or equal to 5×10-6 m²/s at 25° C.

These are, for example, the hexamethyldisiloxane sold under the name “Silbione 70041 V0,65® by the company Rhone Poulenc. This type of product is described in the article by Todd & Byers “Volatile silicone fluids for cosmetics”, Cosmetics and Toiletries, Vol. 91, January 76, pages 27-32.

II. Non-Volatile Silicones

These silicones are mainly constituted by polyalkylsiloxanes, polyarylsiloxanes polyalkylarylsiloxanes and organomodified polysiloxanes, and mixtures thereof. They may be in the form of oils, gums and resins.

Among the polyalkylsiloxanes, mention may be made mainly of linear polydimethylsiloxanes with a viscosity of greater than 5×10⁻⁶ m²/s, and preferably less than 2.6 m²/s, i.e.:
containing trimethylsilyl end groups, such as, for example, and without limitation, the Silbione® oils of series 70047 that are sold by the company Rhone Poulenc, the oil Wacker Belsil DM 60000 from Wacker, or certain Viscasil® products from the company General Electric;
containing trihydroxysilyl end groups, such as the oils from the series 48 V® from the company Rhone Poulenc.

In this class of polyalkylsiloxanes, mention may also be made of the polyalkylsiloxanes sold by the company Goldschmidt under the names Abilwax 9800® and Abilwax 9801®, which are poly(C₁₋₂₀)alkylsiloxanes.

Among the polyalkylarylsiloxanes, mention may be made of linear and/or branched polydimethylphenylsiloxanes and polydimethyldiphenylsiloxanes, with a viscosity from 10⁻⁵ to 5×10² m²/s, for instance:
the oil Rhodorsil® 763 from Rhone-Poulenc,
the Silbione® oils of the 70641 series from Rhone-Poulenc, such as the oils Silbione 70641 V30® and Silbione 70641 V200®,
the product DC 556® Cosmetic Grade Fluid from Dow Corning,
the silicones of the PK series from Bayer, such as PK20®,
the silicones of the PN and PH series from Bayer, such as PN1000® and PH1000®;
certain oils of the SF series from General Electric, such as SF 1250®, SF 1265®, SF 1154® and SF 1023®.

Silicone gums in accordance with the present invention are polydiorganosiloxanes with a high number-average molecular mass of between 200 000 and 1 000 000, used alone or as a mixture in a solvent selected from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Examples include the compounds having the following structures:
poly[(dimethylsiloxane)/(methylvinylsiloxane)] gums,
poly[(dimethylsiloxane)/(diphenylsiloxane)] gums,
poly[(dihydrogenodimethylsiloxane)/(divinylsiloxane)] gums,
poly[(dimethylsiloxane)/(phenylmethylsiloxane)] gums,
poly[(dimethylsiloxane)/(diphenylsiloxane)/(methyl [symbol] vinyl [symbol] siloxane)] gums.

Mention may be made of the gum Mirasil DM 300 000 from the company Rhodia.

Mention may also be made, for example, in a nonlimiting manner, of the following mixtures:

1) mixtures formed from a polydimethylsiloxane hydroxylated at the end of the chain (dimethiconol according to the CTFA nomenclature) and a cyclic polydimethylsiloxane (cyclomethicone according to the CTFA nomenclature), such as the products Q2 1401® or Dow Corning 1501 Fluid sold by the company Dow Corning;

2) mixtures formed from a polydimethylsiloxane gum with a cyclic silicone, such as the product SF 1214 Silicone Fluid® from General Electric, which is a gum SE 30® of MW 500 000 (-Mₙ) dissolved in SF 1202 Silicone Fluid® (decamethylcyclopentasiloxane);

3) mixtures of two PDMSs of different viscosities, especially of a PDMS gum and a PDMS oil, such as the products SF 1236® and CF 1241® from the company General Electric. The product SF 1236® is a mixture of an SE 30® gum defined above, with a viscosity of 20 m²/s., and of an SF 96® oil with a viscosity of 5×106 m²/s. (15% SE 30® gum and 85% SF 96® oil).

The product CF 1241® is a mixture of an SE 30® gum (33%) and of a PDMS (67%), with a viscosity of 10⁻³ m²/s.

The organopolysiloxane resins that may be used in accordance with the invention are crosslinked siloxane systems containing the following units: R₂SiO_{2/2}, RSiO_{3/2} and SiO_{4/2}, in which R represents a hydrocarbon group possessing 1 to 6 carbon atoms or a phenyl group. Among these products, those which are particularly preferred are those in which R denotes a lower (C₁-C₄) alkyl group or a phenyl group.

These resins include the product sold under the name Dow Corning 593® or those sold under the names Silicone Fluid SS 4230 and Silicone Fluid SS 4267 by the company General Electric, which are dimethyl/trimethylpolysiloxanes.

The organomodified silicones in accordance with the present invention are silicones as defined above, comprising, in their general structure, one or more organofunctional groups directly attached to the siloxane chain or attached via a hydrocarbon group.

Examples include the silicones comprising:

a) perfluoro groups such as trifluoroalkyls, for instance those sold by the company General Electric under the names FF.150 Fluorosilicone Fluid® or by the company Shin-Etsu under the names X-22-819®, X-22-82®, X-22-821® and X-22-822®;

b) hydroxyacylamino groups, for instance those described in patent application EP 0 342 834 and in particular the silicone sold by the company Dow Corning under the name Q2-8413®;

c) thiol groups, as in the silicones X 2-8360® from the company Dow Corning or GP 72A® and GP 71® from Genesee;

d) non-quaternized amine groups, such as GP 4 Silicone Fluid® from Genesee, GP 7100® from Genesee, Q2 8220® from Dow Corning, AFL 40® from Union Carbide or the silicone known as Amodimethicone in the CTFA dictionary;

e) carboxylate groups, for instance the products described in patent EP 186 507 from Chisso Corporation;

f) hydroxyl groups, for instance the hydroxyalkyl-functional polyorganosiloxanes described in patent application FR 85 16334, conforming to the formula (XV) below:

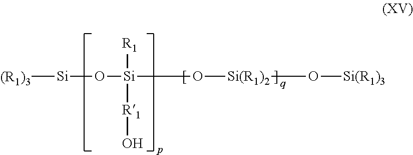

in which:
the groups R₁, which are identical or different, are selected from methyl and phenyl groups, at least 60 mol % of the groups R₁ being methyl;
the group R′₁ is a divalent C₂-C₁₈ hydrocarbon alkylene chain member;
p is between 1 and 30 inclusive;
q is between 1 and 150 inclusive.

Mention may be made especially of the product sold by Dow Corning under the name DC 190;

g) alkoxylated groups, for instance in the silicone copolymer F 755® from SWS Silicones and the products Abilwax 2428®, Abilwax 2434® and Abilwax 2440® from the company Goldschmidt;

h) acyloxyalkyl groups, such as, for example, the polyorganopolysiloxanes described in patent application FR 88 17433, conforming to the formula (XVI) below:

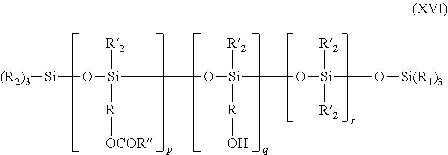

in which:
R₂ denotes methyl, phenyl, OCOR″ or hydroxyl, but only one R₂ per silicon atom may be OH;
R′₂ denotes methyl or phenyl, at least 60 mol % of the entirety of the groups R₂ and R′₂ being methyl;
R″ denotes C₈-C₂₀ alkyl or alkenyl;
R denotes a linear or branched, divalent C₂-C₁₈ hydrocarbon alkylene;
r is between 1 and 120 inclusive;
p is between 1 and 30 inclusive;
q is 0 or is less than 0.5 p, with p+q being between 1 and 30 inclusive:

the polyorganosiloxanes of formula (XVI) can comprise:

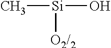

groups in proportions not exceeding 15% of the sum p+q+r;

i) quaternary ammonium groups, as in the products X2 81 08® and X2 81 09® and the product Abil K3270® from the company Goldschmidt;

j) amphoteric or betaine groups, as in the product sold by the company Goldschmidt under the name Abil B 9950®;

k) bisulphite groups, as in the products sold by the company Goldschmidt under the names Abil S 201® and Abil S 255®.

l) polyethyleneoxy and/or polypropyleneoxy groups optionally comprising C₆-C₂₄ alkyl groups, such as the products called dimethicone copolyol as sold by the company Dow Corning under the name DC 1248, or the oils Silwet L 722, L 7500, L 77 and L 711 from the company Union Carbide, and the alkyl (C₁₂) methicone copolyol sold by the company Dow Corning under the name Q2 5200.

According to the invention, it is also possible to use silicones comprising a polysiloxane portion and a portion composed of a non-silicone organic chain, with one of the two portions making up the main chain of the polymer and the other being grafted on the said main chain. These polymers are, for example, described in patent applications EP-A-412 704, EP-A-412 707, EP-A-640 105 and WO 95/00578, EP-A-582 152 and WO 93/23009, and the U.S. Pat. No. 4,693,935, U.S. Pat. No. 4,728,571 and U.S. Pat. No. 4,972,037. These polymers are preferably anionic or nonionic.

Such polymers are, for example, copolymers that may be obtained by radical polymerization from the monomer mixture constituted by:

a) 50% to 90% by weight of tert-butyl acrylate;

b) 0% to 40% by weight of acrylic acid;

c) 5% to 40% by weight of silicone macromer of formula (XVII):

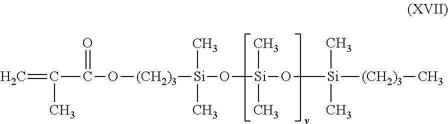

where v is a number from 5 to 700; the percentages by weight are calculated relative to the total weight of the monomers.

Other examples of grafted silicone polymers are, in particular, polydimethylsiloxanes (PDMSs) onto which are grafted, by way of a connecting link of thiopropylene type, mixed polymer units of the poly((meth)acrylic acid) type and of the poly(alkyl (meth)acrylate) type; and polydimethylsiloxanes (PDMSs) onto which are grafted, by way of a connecting link of thiopropylene type, polymer units of the poly(isobutyl (meth)acrylate) type.

According to the invention, all the silicones can also be used in the form of emulsions, nanoemulsions or microemulsions.

The particularly preferred polyorganosiloxanes in accordance with the invention are as follows:
non-volatile silicones selected from the family of polyalkyl [symbol] siloxanes with trimethylsilyl end groups, such as oils having a viscosity of between 0.2 and 2.5 m²/s at 25° C., for instance the oils of the DC200 series from Dow Corning, in particular the one with a viscosity of 60 000 cSt, or of the Silbione 70047 and 47 series, and more particularly the oil 70 047 V 500 000 sold by the company Rhodia Chimie, and polyalkylsiloxanes with dimethylsilanol end groups, such as dimethiconols, or polyalkylarylsiloxanes, for instance the oil Silbione 70641 V 200 sold by the company Rhodia Chimie;
polysiloxanes with amino groups, such as amodimethicones or trimethylsilyl amodimethicones.

The viscosities of the silicones may especially be determined by the standard ASTM D445-97 (viscometry).

When the conditioning agent of the composition according to the invention is a hydrocarbon, it is a linear or branched C8-C30 hydrocarbon.

Among the hydrocarbons which are liquid at ambient temperature and meet this definition, mention may be made in particular of isododecane, isohexadecane and its isomers (such as 2,2,4,4,6,8,8-heptamethylnonane), isoeicosane, isotetracosane, isomers of the said compounds, n-nonadecane, n-dodecane, n-undecane, n tridecane, n-pentadecane, and the mixtures of these hydrocarbons.

Use is preferably made according to the invention of isododecane or an isomer thereof.

When the conditioning agent is a fatty alcohol, this alcohol is a linear or branched, saturated or unsaturated C₈-C₃₀ alcohol. Such alcohols may include, for example, 2-butyloctanol, lauryl alcohol, 2 octyldodecanol, oleyl alcohol, isocetyl alcohol, isostearyl alcohol, behenyl alcohol and mixtures thereof.

When the conditioning agent is a fatty ester, this ester may be either an ester of a C₈-C₃₀ fatty acid and C₁-C₃₀ alcohol, and especially an ester of C₈-C₃₀ fatty acid and C₈-C₃₀ fatty alcohol, or an ester of a C₁-C₇ acid or diacid and a C₈-C₃₀ fatty alcohol.

These esters may include, for example, ethyl, isopropyl, 2 ethylhexyl and 2-octyldecyl palmitate, isopropyl, butyl, cetyl and 2 octyldecyl myristate, butyl and hexyl stearate, hexyl and 2 hexyldecyl laurate, isononyl isononanoate, dioctyl malate, myristyl myristate, cetyl palmitate, and mixtures thereof.

The ceramides or ceramide analogues, such as glycoceramides, that can be used as conditioning agent in the compositions according to the invention are known per se and are natural or synthetic molecules that can correspond to the general formula (XVIII) below:

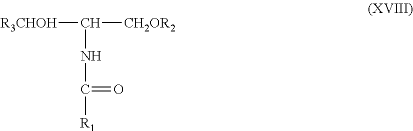

in which:
R₁ denotes a linear or branched, saturated or unsaturated alkyl group which derives from C₁₄-C₃₀ fatty acids, it being possible for this group to be substituted in alpha position by a hydroxyl group, or in omega position by a hydroxyl group which is esterified with a saturated or unsaturated C₁₆-C₃₀ fatty acid;
R² denotes a hydrogen atom or a (glycosyl)n, (galactosyl)m or sulphogalactosyl group, where n is an integer from 1 to 4 and m is an integer from 1 to 8;
R₃ denotes a C₁₅-C₂₆ hydrocarbon group which is saturated or unsaturated in alpha position, it being possible for this group to be substituted by one or more C₁-C₁₄ alkyl groups;

with the proviso that, in the case of natural ceramides or glycoceramides, R₃ may also denote a C₁₅-C₂₆ alpha-hydroxyalkyl group, the hydroxyl group being optionally esterified with a C₁₆-C₃₀ alpha-hydroxyacid.

The ceramides that are preferred in the context of the present invention are those described by Downing in Arch. Dermatol., Vol. 123, 1381-1384, 1987, or those described in French patent FR 2 673 179.

The ceramide or ceramides that are more particularly preferred according to the invention are the compounds for which R1 denotes a saturated or unsaturated alkyl derived from C₁₆-C₂₂ fatty acids; R₂ denotes a hydrogen atom; and R₃ denotes a saturated linear C₁₅ group.

Such compounds are, for example:
N-linoleoyldihydrosphingosine,
N-oleoyldihydrosphingosine,
N-palmitoyldihydrosphingosine,
N-stearoyldihydrosphingosine,
N-behenoyldihydrosphingosine,

or mixtures of these compounds.

More preferably still, ceramides are used for which R₁ denotes a saturated or unsaturated alkyl group derived from fatty acids; R₂ denotes a galactosyl or sulphogalactosyl group; and R₃ denotes a CH═CH—(CH₂)₁₂—CH₃ group.

Examples include the product composed of a mixture of these compounds and sold under the trade name Glycocer by the company Waitaki International Biosciences.

Among all of these conditioning agents, preference is given to the use of one or more conditioning agents selected from silicones such as organosiloxanes and cationic polymers.

The cosmetic composition according to the invention preferably comprises at least 0.01% by weight and more preferentially from 0.05% to 10% by weight of conditioning agent(s), relative to the total weight of the composition.

The cosmetic composition according to the invention may have a pH of between 3 and 10 and preferentially between 5 and 7. This pH may be adjusted by means of acidifying and basifying agents conventionally used in cosmetics.

The cosmetic composition used according to the invention may further comprise one or more adjuvants which are common in the cosmetic field, such as anti-hair-loss agents, oxidizing agents, vitamins and provitamins, including panthenol, vegetable, animal, mineral or synthetic oils, waxes, sunscreen agents, coloured or non-coloured, organic or inorganic pigments, dyes, pearlizing and opacifying agents, sequestrants, plasticizers, solubilizers, antioxidants, hydroxyacids, fragrances, antidandruff agents other than the compounds (i) and (ii) as defined above, preservatives and mixtures thereof.

The amounts of these various adjuvants are those conventionally used in the fields under consideration.

Of course, a person skilled in the art will take care to select the optional compound(s) to be added to the composition according to the invention, such that the advantageous properties intrinsically associated with the composition in accordance with the invention are not, or are not substantially, adversely affected by the envisaged addition.

The invention further provides a cosmetic antidandruff treatment method intended to eliminate and/or reduce dandruff, and especially that caused by yeasts of the genus Malassezia, characterized in that it comprises applying a composition according to the invention to the hair and the scalp. The composition may then optionally be rinsed out with water.

The invention further provides for the use of the combination of at least one compound (i) as defined above, and at least one compound (ii) as defined above, in a weight ratio of the amount of compound(s) (i) to the amount of compound(s) (ii) of greater than or equal to 0.5, preferably greater than or equal to 0.7, more preferably greater than or equal to 1, as an antidandruff agent, particularly in a composition according to the invention. Furthermore, the use of the said combination makes it possible to eliminate and/or reduce dandruff on the hair and the scalp, limiting the irritation and itching of the scalp.

The examples which follow are intended to illustrate the invention, but without having any limiting character.

### Examples 1-2

### Antidandruff Gels

The following compositions were prepared according to the following tables. The amounts are indicated as weight percentages of active material (AM) relative to the total weight of each composition.

### Example 1

### Aqueous-Alcoholic Gel (ZnPt)

| Composition 1 | weight % of AM |
|---|---|
| Crosslinked carboxyvinyl homopolymer | 0.3 |
| (Carbopol Ultrez 10 sold by the company Lubrizol) | |
| 96° ethyl alcohol | 17.4 |
| Ellagic acid | 0.5 |
| Zinc salt of 1-hydroxy-2(1H)-pyridinethione, at | 0.25 |
| 48% by weight in aqueous dispersion (Zinc Omadine | |
| Pyrithione 48% DSP Cosm sold by the company Arch | |
| Chemicals) | |
| Water | qs 100 |

The weight ratio of the amount of compound (i)/(ii) is equal to 2.

The gel obtained is stable over time and has satisfactory rheology.

Applied to the scalp and the hair, this gel exhibits good cosmetic performance (no adverse alteration of the feel of the hair) and a satisfactory antidandruff effect in combination with high skin tolerance (notable reduction in the phenomena of irritation and itching).

### Example 2

### Aqueous-Alcoholic Gel (Piroctone Olamine)

| Composition 2 | weight % of AM |
|---|---|
| Crosslinked carboxyvinyl homopolymer | 0.3 |
| (Carbopol Ultrez 10 sold by the company Lubrizol) | |
| 96° ethyl alcohol | 17.4 |
| Ellagic acid | 0.5 |
| Piroctone olamine (Octopirox sold by the company | 0.05 |
| Clariant) | |
| Water | qs 100 |

The weight ratio of the amount of compound (i)/(ii) is equal to 10.

The gel obtained is stable over time and has satisfactory rheology.

Applied to the scalp and the hair, this gel exhibits good cosmetic performance (no adverse alteration of the feel of the hair) and a satisfactory antidandruff effect in combination with high skin tolerance (notable reduction in the phenomena of irritation and itching).

### Examples 3-5

### Antidandruff Shampoos

The following compositions were prepared according to the following tables. The amounts are indicated as weight percentages of active material (AM) relative to the total weight of each composition.

### Example 3

### Antidandruff Shampoo (ZnPt)

| Composition 3 | weight % of AM |
|---|---|
| Sodium benzoate | 0.5 |
| Sodium lauryl ether sulphate containing | 11 |
| 2.2 mol of ethylene oxide, at 26% by | |
| weight in aqueous solution (Texapon | |
| AOS 225 UP sold by the company Cognis) | |
| Sodium lauryl sulphate at 34.5% by | 4 |
| weight in aqueous solution (Texapon LS | |
| 35 sold by the company Cognis) | |
| Hydroxyethylcellulose crosslinked with | 0.19 |
| epichlorohydrin and quaternized with | |
| trimethylamine, in aqueous solution at | |
| 95% by weight of active material | |
| (Polyquat 400KC sold by the company KCI) | |
| Zinc salt of 1-hydroxy-2(1H)- | 1 |
| pyridinethione, at 48% by weight in | |
| aqueous dispersion (Zinc Omadine | |
| Pyrithione 48% DSP Cosm sold by the | |
| company Arch Chemicals) | |
| Ellagic acid | 1 |
| Ethylene glycol distearate | 1 |
| Salicylic acid | 0.2 |
| Coconut monoisopropanolamide | 2 |
| (Comperlan 100 sold by the company Cognis) | |
| Polydimethylsiloxane (Mirasil DM | 0.4 |
| 300 000 sold by the company Rhodia) | |
| Fragrance | 0.5 |
| Carboxyvinyl polymer (Carbopol 980 | 0.3 |
| sold by the company Lubrizol) | |
| Sodium chloride | 1.2 |
| Water | qs 100 |

The weight ratio of the amount of compound (i)/(ii) is equal to 1.

The shampoo obtained is stable over time and has satisfactory rheology.

Applied to the hair and the scalp, and rinsed out, this shampoo exhibits good cosmetic performance (good feel of the hair) and a satisfactory antidandruff effect in combination with good skin tolerance (notable reduction in the phenomena of irritation and itching).

### Example 4

### Antidandruff Shampoo (Piroctone Olamine)

| Composition 4 | weight % of AM |
|---|---|
| Cocamidopropylbetaine at 38% by | 2.4 |
| weight in aqueous solution (Tego betaine | |
| F50 sold by the company Evonik Goldschmidt) | |
| Sodium lauryl ether sulphate containing | 15.55 |
| 2.2 mol of ethylene oxide, at 26% by | |
| weight in aqueous solution (Texapon | |
| AOS 225 UP sold by the company Cognis) | |
| Ellagic acid | 1 |
| Piroctone olamine (octopirox sold by the | 0.5 |
| company Clariant) | |
| Polydimethylsiloxane 500 000 cSt | 2 |
| (molecular weight: 250 000) (Silbione | |
| 70047V500000 sold by the company Bluestar) | |
| Monoisopropanolamine of coconut acids | 0.24 |
| (Empilan CIS sold by the company Huntsman) | |
| Carboxyvinyl polymer (Carbopol 980 | 0.3 |
| sold by the company Lubrizol) | |
| 1-(hexadecyloxy)-2-octadecanol/cetyl | 2.5 |
| alcohol mixture (Mexanyl GY sold by | |
| the company Chimex) | |
| Propylene glycol | 0.1 |
| Sodium benzoate | 0.5 |
| Salicylic acid | 0.2 |
| Glycerol | 2 |
| Sodium chloride | 1.1 |
| Fragrance | 0.5 |
| Water | qs 100 |

The weight ratio of the amount of compound (i)/(ii) is equal to 2.

The shampoo obtained is stable over time and has satisfactory rheology.

Applied to the hair and the scalp, and rinsed out, this shampoo exhibits good cosmetic performance (good feel of the hair) and a satisfactory antidandruff effect in combination with good skin tolerance (notable reduction in the phenomena of irritation and itching).

### Example 5

### Antidandruff Shampoo (SeS₂)

| Composition 5 | weight % of AM |
|---|---|
| Sodium hypochlorite at 14% by weight in | 0.06 |
| aqueous solution | |
| Sodium lauryl ether sulphate containing | 14.8 |
| 2.2 mol of ethylene oxide, at 26% by | |
| weight in aqueous solution (Texapon | |
| AOS 225 UP sold by the company Cognis) | |
| Cocobetaine at 30% by weight in aqueous | 2.4 |
| solution (Dehyton AB 30 sold by the company | |
| Cognis) | |
| Selenium disulphide as micronized powder | 1 |
| (Selenium sulfide USP micronized sold by the | |
| company Eskay Fine Chemicals) | |
| Ellagic acid | 1 |
| Ethoxylated (20 mol of ethylene oxides) and | 0.7 |
| propoxylated (5 mol of propylene oxide) cetyl | |
| alcohol (Procetyl AWS LQ sold by the company | |
| Croda) | |
| Ethoxylated lauryl alcohol containing 2.5 | 0.9 |
| mol of ethylene oxide (Arlypon F sold by | |
| the company Cognis) | |
| Mixture of linear (C₁₈/C₂₀/C₂₂) alcohols | 1.5 |
| (Nafol 1822 C sold by the company Sasol) | |
| Distearyl ether (Cutina STE sold by the company | 1.5 |
| Cognis) | |
| Polydimethylsiloxane (Mirasil DM | 2 |
| 300 000 sold by the company Rhodia) | |
| Carboxyvinyl polymer (Carbopol 980 | 0.4 |
| sold by the company Lubrizol) | |
| Citric acid•1 H₂O | 0.3 |
| Sodium chloride | 0.35 |
| Fragrance, preservative | 0.7 |
| Water | qs 100 |

The weight ratio of the amount of compound (i)/(ii) is equal to 1.

The shampoo obtained is stable over time and has satisfactory rheology.

Applied to the hair and the scalp, and rinsed out, this shampoo exhibits good cosmetic performance (good feel of the hair) and a satisfactory antidandruff effect in combination with good skin tolerance (notable reduction in the phenomena of irritation and itching).

Although the invention has been described with respect to preferred embodiments, it is to be also understood that it is not to be so limited since changes and modifications can be made therein which are within the full scope of this invention as detailed by the following claims.

What is claimed is:

1. Cosmetic composition comprising:
(i) one or more compounds selected from ellagic acid, its ethers and the salts of ellagic acid and its ethers, and
(ii) one or more compounds selected from pyrithione salts and selenium (poly)sulphides,
in a weight ratio of the amount of compound(s) (i) to the amount of compound(s) (ii) of greater than or equal to 0.5.

2. Composition according to claim 1, characterized in that the salt(s) are selected from alkali metal or alkaline-earth metal salts, especially the sodium, potassium, calcium or magnesium salts.

3. Composition according to claim 1, characterized in that the ether or ethers are selected from mono-, di-, tri- or polyethers obtained by etherification of one or more hydroxyl groups of the ellagic acid to one or more groups OR, R being selected from C₂-C₂₀ alkyl groups, polyoxyalkylene groups and groups derived from one or more mono- or polysaccharides.

4. Composition according to claim 1, characterized in that the compound (i) is ellagic acid or a salt thereof.

5. Composition according to claim 1, characterized in that it comprises from 0.01% to 10% by weight, preferably from 0.1% to 5% by weight, and more preferably from 0.2% to 2% by weight of compound(s) (i), relative to the total weight of the composition.

6. Composition according to claim 1, characterized in that the pyrithione salt or salts are selected from monovalent metal salts and divalent metal salts such as sodium, calcium, magnesium, barium, strontium, zinc, cadmium, tin and zirconium salts.

7. Composition according to claim 1, characterized in that the pyrithione salt is a zinc salt.

8. Composition according claim 1, characterized in that the selenium (poly)sulphide or (poly)sulphides are selected from selenium disulphide and the selenium polysulphides of formula SeₓS_{y} in which x and y are numbers such that x+y=8.

9. Composition according to claim 1, characterized in that the selenium (poly)sulphide is selenium disulphide.

10. Composition according to claim 1, characterized in that it comprises from 0.01% to 10% by weight, preferably from 0.02% to 5% by weight, and more preferably from 0.04% to 2% by weight of compound(s) (ii), relative to the total weight of the composition.

11. Composition according to claim 1, characterized in that the weight ratio of the amount of compound(s) (i) to the amount of compound(s) (ii) is greater than or equal to 0.7, and more preferably greater than or equal to 1.

12. Composition according to claim 1, characterized in that it comprises one or more thickeners, one or more surfactants selected from anionic, nonionic, and/or amphoteric or zwitterionic surfactants, and/or one or more conditioning agents, preferably selected from silicones and cationic polymers.

13. Composition according to claim 1, characterized in that it comprises one or more additives selected from anti-hair loss agents, oxidizing agents, vitamins and provitamins, including panthenol, vegetable, animal, mineral or synthetic oils, waxes, sunscreen agents, coloured or non-coloured, organic or inorganic pigments, dyes, pearlizing and opacifying agents, sequestrants, plasticizers, solubilizers, antioxidants, hydroxyacids, fragrances, antidandruff agents other than the compounds (i) and (ii) as defined in claim 1, preservatives, acidifying agents, basifying agents, and mixtures thereof.
